(19) United States
(12) Patent Application Publication
(10) Pub. No.: US 2024/0141405 A1
(43) Pub. Date: May 2, 2024

(54) COMPOSITION FOR PREDICTING OR DIAGNOSING EARLY-STAGE DIFFUSE GASTRIC CANCER, COMPRISING PREPARATION FOR MEASURING LEVEL OF PEPSINOGEN II

(71) Applicant: Seoul National University Hospital, Seoul (KR)

(72) Inventor: Nayoung Kim, Seongnam-Si (KR)

(21) Appl. No.: 18/015,207

(22) PCT Filed: Sep. 17, 2020
(86) PCT No.: PCT/KR2020/012575

§ 371 Date: Oct. 2, 2023

(30) Foreign Application Priority Data
Jul. 13, 2020 (KR) 10-2020-0086191
Sep. 9, 2020 (KR) 10-2020-0115244

(51) Int. Cl. C12Q 1/04; G01N 33/574

(52) U.S. Cl. CPC C12Q 1/04; G01N 33/57446

(57) ABSTRACT

The composition, kit and information provision method, according to an aspect of the present invention, may predict or diagnose early-stage diffuse-type gastric cancer with high sensitivity and specificity when the level of serum pepsinogen II is equal to or greater than 20 μg/L, and further, may classify the risk of early-stage diffuse-type gastric cancer in a subject on the basis of whether the level of serum pepsinogen II is equal to or greater than 20 μg/L and whether the status of Helicobacter pylori (HP) is positive, and thus, the composition, comprising a preparation capable of measuring the level of serum pepsinogen II, or, along with said preparation, a preparation capable of detecting the status of HP, the kit and the information provision method using same have an excellent effect of being capable of being usefully employed as non-invasive means which may be carried out prior to an endoscopy.

### TECHNICAL FIELD

This application claims the benefit of priority of the prior Korean Patent Application No. 10-2020-0086191 filed on Jul. 13, 2020, the entire contents of which are incorporated herein by reference. This application also claims the benefit of priority of the prior Korean Patent Application No. 10-2020-0115244 filed on Sep. 9, 2020, the entire contents of which are incorporated herein by reference.

In the present specification, a composition, kit and information provision method for discovering pepsinogen II as a biomarker for predicting or diagnosing early-stage diffuse gastric cancer and enabling predicting or diagnosing early-stage diffuse gastric cancer according to the level of pepsinogen II measured in a subject are disclosed.

### BACKGROUND ART

The prevalence rate of gastric cancer (GC) has substantially declined over the past 40 years worldwide, but gastric cancer is still the fifth most frequently diagnosed malignant tumor and the third leading cause of cancer death worldwide. Above all, while the incidence rate is significantly increased in East Asia including Korea, the incidence rate is generally low in North America and Northern Europe [Bray F, Ferlay J, Soerjomataram I, Siegel R L, Torre L A, Jemal A. Global cancer statistics 2018: GLOBOCAN estimates of incidence and mortality worldwide for 36 cancers in 185 countries. CA Cancer J Clin 2018; 68:394-424]. Since gastric cancer (GC) is diagnosed at an advanced stage in many cases due to its non-specific symptoms, a screening strategy to detect gastric cancer at an early and more treatable stage has emerged as an important issue. The Korean National Cancer Screening Program for gastric cancer centered on endoscopy began in the late 20th century and has significantly reduced cancer-related mortality [Jun J K, Choi K S, Lee H Y, et al. Effectiveness of the Korean National Cancer Screening Program in reducing gastric cancer mortality. Gastroenterology 2017; 152:1319-1328].

As an alternative method, a non-invasive mass screening method using serum pepsinogen (sPG) is gaining popularity in Japan. Serum pepsinogen I (sPGI) and serum pepsinogen II (sPGII) are produced in different parts of the gastric mucosa [Samloff I M. Pepsinogens, pepsins, and pepsin inhibitors. Gastroenterology 1971; 60:586-604]. In other words, while serum pepsinogen I is secreted only from chief cells in the fundic glands, serum pepsinogen II is secreted not only from the fundic glands but also from the pyloric glands of the antrum and the duodenal mucosa. According to previous studies, low sPGI (<70 μg/L) and low sPGI/II ratio (<3) are indicators of advanced atrophic gastritis, and this is associated with a high risk of gastric cancer [Samloff I M. Pepsinogens, pepsins, and pepsin inhibitors. Gastroenterology 1971; 60:586-604][Kodoi A, Yoshihara M, Sumii K, Haruma K, Kajiyama G. Serum pepsinogen in screening for gastric cancer. J Gastroenterol 1995; 30:452-460]. When the gastric mucosa is inflamed by Helicobacter pylori (HP) infection, the production of serum pepsinogen I is decreased in the atrophic mucosa but serum pepsinogen II is increased. Hence, the sPGI/II ratio further decreases in association with low sPGI and increased sPGII in advanced atrophic gastritis [Samloff I M, Varis K, Ihamaki T, Siurala M, Rotter J I. Relationships among serum pepsinogen I, serum pepsinogen II, and gastric mucosal histology: a study in relatives of patients with pernicious anemia. Gastroenterology 1982; 83:204-209]. However, although these criteria can be applied to intestinal-type GC (IGC) following the Correa cascade, questions remain as to whether these criteria can be applied to diffuse-type GC (DGC) with a different carcinogenic mechanism [Uemura N, Okamoto S, Yamamoto S, et al. Helicobacter pylori infection and the development of gastric cancer. N Engl J Med 2001; 345:784-789]. In addition, in the case of atrophic gastritis, serum pepsinogen I and the ratio (sPGI/II) of serum pepsinogen I to serum pepsinogen II are low, so the role of serum pepsinogens has been controversial in applying the Japanese standard cutoff value to gastric cancer screening in Korea or other countries including Korea although serum pepsinogen is used as an effective biomarker for gastric cancer screening in Japan. In other words, there has been a problem that the low specificity of serum pepsinogen I has lower predictive ability than that in previous studies [Kang J M, Kim N, Yoo J Y, et al. The role of serum pepsinogen and gastrin test for the detection of gastric cancer in Korea. Helicobacter 2008; 13:146-156]. The inventors of the present invention have reported that the high-risk OLGA (operative link on gastric atrophy) and OLGIM (operative link on gastric intestinal metaplasia) stages are important predictive markers for not only intestinal-type gastric cancer but also diffuse-type gastric cancer [Yun C Y, Kim N, Lee J, et al. Usefulness of OLGA and OLGIM system not only for intestinal type but also for diffuse type of gastric cancer, and no interaction among the gastric cancer risk factors. Helicobacter 2018; 23:e12542]. Diffuse-type gastric cancer in Korea accounts for a relatively high proportion (42.1%) of all gastric cancer cases, which is quite different from that in Japanese studies [Kang J M, Kim N, Yoo J Y, et al. The role of serum pepsinogen and gastrin test for the detection of gastric cancer in Korea. Helicobacter 2008; 13:146-156]. Hence, if there is a useful biomarker for diffuse-type gastric cancer, the biomarker would be greatly useful in Korea. So far, there have been studies suggesting association between a high titer of serum pepsinogen II and diffuse-type gastric cancer [Kikuchi S, Wada 0, Miki K, et al. Serum pepsinogen as a new marker for gastric carcinoma among young adults. Research Group on Prevention of Gastric Carcinoma among Young Adults. Cancer 1994; 73:2695-2702][Yanaoka K, Oka M, Yoshimura N, et al. Risk of gastric cancer in asymptomatic, middle-aged Japanese subjects based on serum pepsinogen and Helicobacter pylori antibody levels. Int J Cancer 2008; 123:917-926][Ito M, Yoshihara M, Takata S, et al. Serum screening for detection of high-risk group for early-stage diffuse type gastric cancer in Japanese. J Gastroenterol Hepatol 2012; 27:598-602].

It is known that serum pepsinogen II level is associated with histological changes reflecting the degree of inflammation due to Helicobacter pylori infection in the gastric mucosa. In other words, the serum pepsinogen II level is higher in non-atrophic gastritis associated with Helicobacter pylori (HP) and lower in atrophic gastritis, and the serum pepsinogen II level can be reversed by eradication of Helicobacter pylori (HP) [Plebani M, Basso D, Cassaro M, et al. Helicobacter pylori serology in patients with chronic gastritis. Am J Gastroenterol 1996; 91:954-958][Mårdh E, Mårdh S, Mårdh B, Borch K. Diagnosis of gastritis by means of a combination of serological analyses. Clin Chim Acta 2002; 320:17-27.][Pilotto A, Di Mario F, Franceschi M, et al. Cure of Helicobacter pylori infection in the elderly: effects of eradication on gastritis and serological markers. Aliment Pharmacol Ther 1996; 10:1021-1027.][Kawai T, Miki K, Ichinose M, et al. Changes in evaluation of the pepsinogen test result following Helicobacter pylori eradication therapy in Japan. Inflammopharmacology 2007; 15:31-35.]. A previous study has also revealed that the expression level of pepsinogen II significantly decreases in malignant gastric mucosa and the positive rate of pepsinogen II expression is regulated by Helicobacter pylori infection [Ning P F, Liu H J, Yuan Y. Dynamic expression of pepsinogen C in gastric cancer, precancerous lesions and Helicobacter pylori associated gastric diseases. World J Gastroenterol 2005; 11:2545-2548].

Hence, it is necessary to redefine the role of serum pepsinogen in relation to its usefulness as a biomarker of diffuse-type gastric cancer to stratify the development of gastric cancer, particularly patients requiring endoscopy. This will contribute to a further decrease in gastric cancer-related mortality in East Asia, including China and Korea. Under this background, the present inventors have noted that an attractive biomarker for gastric cancer has not been established so far, studied the role of serum pepsinogens in the development of gastric cancer focusing on the ambiguous role of serum pepsinogen II that has not been reported frequently. As a result, the inventors found out that a serum pepsinogen II level of ≥20 μg/L is associated with the development of diffuse-type gastric cancer, particularly early-stage diffuse-type gastric cancer in subjects who are not elderly in a large cohort, and thus completed the present invention.

### SUMMARY OF INVENTION

### Technical Problem

The present inventors have conducted studies on a total of about 3,000 subjects classified into a gastric dysplasia group, a gastric cancer group, and a control group to discover serum pepsinogen II as a biomarker of gastric cancer, specifically diffuse-type gastric cancer, and verified that the level of pepsinogen II measured in a subject's sample can predict or diagnose the risk of early-stage diffuse-type gastric cancer with high sensitivity and specificity, and can classify the risk of diffuse-type gastric cancer in a subject through the subject's Helicobacter pylori infection history in addition to the level of pepsinogen II.

Accordingly, in an aspect, an object of the present invention is to provide a biomarker for predicting or diagnosing early-stage diffuse-type gastric cancer.

In another aspect, an object of the present invention is to provide a biomarker for predicting or diagnosing early-stage diffuse-type gastric cancer in a subject under the age of 40.

In still another aspect, an object of the present invention is to provide a biomarker for predicting or diagnosing the risk of early-stage diffuse-type gastric cancer in a subject.

### Solution to Problem

In an aspect, the present invention provides a composition for predicting or diagnosing early-stage diffuse-type gastric cancer, comprising a preparation for measuring the level of pepsinogen II (PGII) in a sample of a subject under the age of 40.

In another aspect, the present invention provides a composition for predicting or diagnosing early-stage diffuse-type gastric cancer, comprising a preparation for measuring the level of pepsinogen II in serum of a subject under the age of 40 and a preparation for detecting Helicobacter pylori.

In still another aspect, the present invention provides a kit for predicting or diagnosing early-stage diffuse-type gastric cancer, comprising the compositions described above.

In still another aspect, the present invention provides an information provision method for predicting or diagnosing early-stage diffuse-type gastric cancer, comprising measuring the level of pepsinogen II in a sample of a subject under the age of 40.

### Advantageous Effects of Invention

In an aspect of the present invention, it has been confirmed that the level of serum pepsinogen II is higher in subjects with early-stage diffuse-type gastric cancer, particularly in subjects with early-stage diffuse-type gastric cancer under the age of 40, compared to controls, and a subject with early-stage diffuse-type gastric cancer has a history of Helicobacter pylori infection (positive HP status) currently or in the past, and thus found out that early-stage diffuse-type gastric cancer can be predicted or diagnosed with high sensitivity and specificity if the serum pepsinogen II level is equal to or greater than 20 μg/L, and further whether the serum pepsinogen II level is equal to or greater 20 μg/L and whether the HP status is positive are criteria for classifying the risk of early-stage diffuse-type gastric cancer in a subject. From this, it can be seen that a composition comprising a preparation capable of measuring the level of serum pepsinogen II or comprising the preparation capable of measuring the level of serum pepsinogen II and a preparation capable of detecting the Helicobacter pylori (HP) status, and a kit comprising the composition have an excellent effect of being capable of being usefully employed as non-invasive means which may be carried out prior to an endoscopy.

### BRIEF DESCRIPTION OF DRAWINGS

FIG. 1 is a diagram illustrating classification of enrolled subjects into a gastric cancer group, a gastric dysplasia group, and a control group according to an aspect of the present invention. Abbreviations in FIG. 1 are as follows: MALT, mucosa-associated lymphoid tissue; and GIST, gastrointestinal stromal tumor.

FIGS. 2A to 2D are graphs illustrating the relation between the levels of serum pepsinogens (sPGs) in subjects and the Helicobacter pylori (HP) status in the OLGA/OLGIM stage according to an aspect of the present invention. FIGS. 2A and 2B illustrate the results of comparing the levels of serum pepsinogens (sPGs) associated with the Helicobacter pylori (HP) status to the histological characteristics, in which subjects with a positive HP status have higher levels of serum pepsinogen I (sPGI) (FIG. 2A) and serum pepsinogen II (sPGII) (FIG. 2B) and lower sPGI/II ratios (FIG. 2C) than subjects with a negative HP status (all p<0.001). FIG. 2D illustrates the relation between the levels of serum pepsinogens (sPGs) and the OLGA and OLGIM stages, in which the OLGA and OLGIM stages are higher in subjects with a positive HP status than in subjects with a negative HP status (FIG. 2D). In FIGS. 2A to 2D, data are presented as numbers (%) or median±standard error, and abbreviations are as follows: OLGA, operative link on gastritis atrophy; OLGIM, operative link on gastric intestinal metaplasia.

FIGS. 3A to 3D and 4 relate to the results of calculating the AUC (area under the curve) to confirm the usefulness of serum pepsinogen II (sPGII) as a biomarker for predicting or diagnosing early-stage diffuse-type gastric cancer according to an embodiment of the present invention, and are graphs illustrating the receiver operating characteristic curve (ROC) and corresponding AUC of serum pepsinogen II (sPGII) for diagnosing gastric cancer (GC). According to FIGS. 3A and 3B, the AUC of serum pepsinogen II (sPGII) does not have significant sensitivity and specificity for all gastric cancer (GC) cases (FIG. 3A) and all diffuse-type gastric cancer (DGC) cases (FIG. 3B). On the other hand, according to FIGS. 3C and 3D, when the early-stage diffuse-type gastric cancer subjects are classified based on the age of 40, serum pepsinogen II (sPGII) has significantly higher diagnostic power (AUC 0.766, sensitivity 75.0%, specificity 74.2%) in early-stage diffuse-type gastric cancer subjects under the age of 40 (FIG. 3D) compared to early-stage diffuse-type gastric cancer subjects over the age of 40 (FIG. 3C). According to FIG. 4, it can be seen that early-stage diffuse-type gastric cancer can be diagnosed with higher sensitivity and specificity when the subject is a female under the age of 40 (AUC 0.824, sensitivity 81.3%, specificity 78.1%) (FIG. 4). Abbreviations in FIGS. 3A to 3D and 4 are as follows: AUC, area under the curve; sPG, serum pepsinogen; DGC, diffuse-type gastric cancer.

### DESCRIPTION OF EMBODIMENTS

The present invention is described in details hereinafter.

In an aspect of the present invention, the “prediction of early-stage diffuse-type gastric cancer” may mean predicting or diagnosing whether there is a possibility of developing early-stage diffuse-type gastric cancer in a subject, whether the possibility of developing early-stage diffuse-type gastric cancer is relatively high, what the causative factor of early-stage diffuse-type gastric cancer is, or whether early-stage diffuse-type gastric cancer has already been developed. In an aspect of the present invention, the “diagnosis of early-stage diffuse-type gastric cancer” means confirming the presence or characteristics of a pathological state of a subject, and for the purpose according to an aspect of the present invention, diagnosis may mean confirming whether early-stage diffuse-type gastric cancer has been developed. The composition, kit or method according to an aspect of the present invention can be used to delay the time of onset or prevent the onset through special and appropriate management for any specific patient with high risk of developing early-stage diffuse-type gastric cancer. The composition, kit or method according to an aspect of the present invention can be used clinically to determine treatment by diagnosing early-stage diffuse-type gastric cancer at an early stage and selecting the most appropriate treatment method.

In an aspect of the present invention, the unit or standard of “level of pepsinogen II” or “detected amount of pepsinogen II” for predicting or diagnosing early-stage diffuse-type gastric cancer may vary depending on the method for measuring the level of pepsinogen II, specifically, serum pepsinogen II. More specifically, in the case of measuring or detecting the level of pepsinogen II through a latex-enhanced turbidimetric immunoassay (L-TIA; HBi Corp, Seoul, Korea, imported from Shima Laboratories, Tokyo, Japan), it may be predicted or diagnosed as early-stage diffuse-type gastric cancer when the level of pepsinogen II is equal to or greater than 20 μg/L, but the level of pepsinogen II is not limited to the above value since it may be predicted or diagnosed as early-stage diffuse-type gastric cancer if the level of pepsinogen II measured according to another measurement method is a level corresponding to the pepsinogen II level of equal to or greater than 20 μg/L measured according to the latex-enhanced turbidimetric immunoassay.

In an aspect, the present invention provides a composition for predicting or diagnosing early-stage diffuse-type gastric cancer, comprising a preparation for measuring the level of pepsinogen II (PGII) in a sample of a subject under the age of 40.

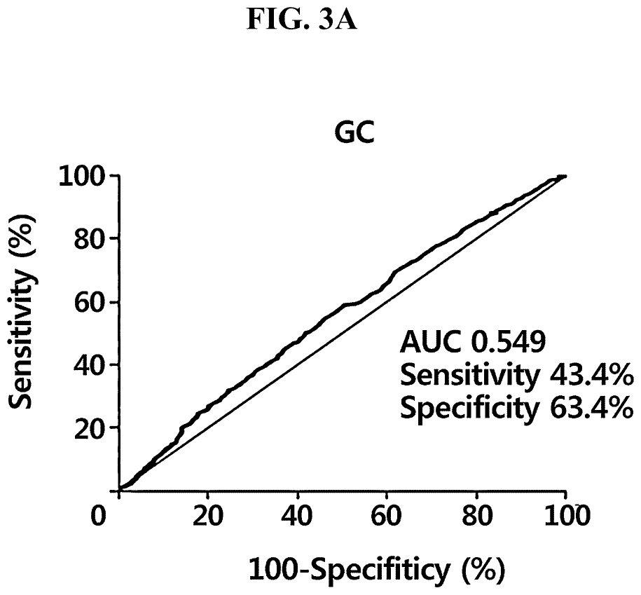

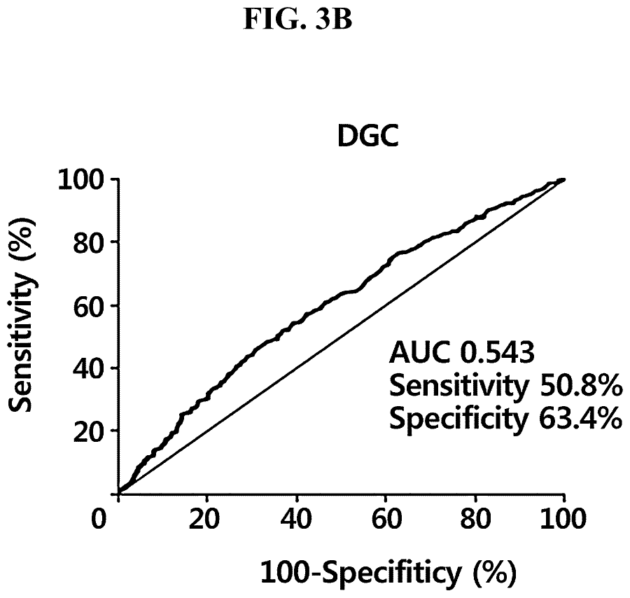

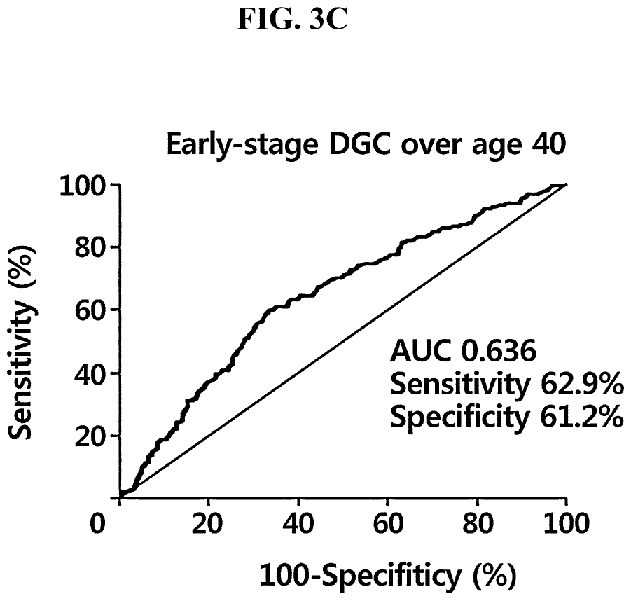

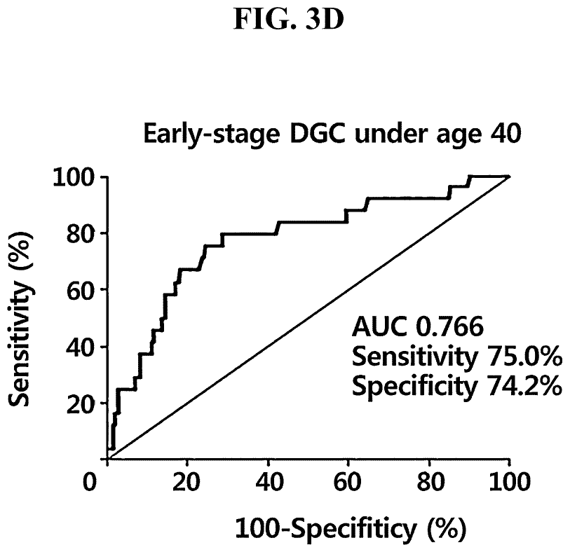

The composition according to an aspect of the present invention may contain a preparation for measuring the level of pepsinogen II, specifically a preparation for measuring the level of serum pepsinogen II (sPGII). The level of serum pepsinogen II is associated with histological changes reflecting the degree of inflammation due to Helicobacter pylori infection in the gastric mucosa, the level of serum pepsinogen II is higher in non-atrophic gastritis associated with Helicobacter pylori (HP) and lower in atrophic gastritis, and the level of serum pepsinogen II may be reversed by eradication of Helicobacter pylori (HP) [Plebani M, Basso D, Cassaro M, et al. Helicobacter pylori serology in patients with chronic gastritis. Am J Gastroenterol 1996; 91:954-958][Mårdh E, Mårdh S, Mårdh B, Borch K. Diagnosis of gastritis by means of a combination of serological analyses. Clin Chim Acta 2002; 320:17-27.][Pilotto A, Di Mario F, Franceschi M, et al. Cure of Helicobacter pylori infection in the elderly: effects of eradication on gastritis and serological markers. Aliment Pharmacol Ther 1996; 10:1021-1027.][Kawai T, Miki K, Ichinose M, et al. Changes in evaluation of the pepsinogen test result following Helicobacter pylori eradication therapy in Japan. Inflammopharmacology 2007; 15:31-35.]. According to an embodiment of the present invention, the level of serum pepsinogen II was high in subjects with early-stage diffuse-type gastric cancer (Example 4 and Table 2), and it has been confirmed that early-stage diffuse-type gastric cancer can be predicted or diagnosed with high sensitivity and specificity when the level of serum pepsinogen II in a subject is equal to or greater than 20 μg/L (Examples 6 and 7, Table 3 and FIG. 3).

The preparation for measuring the level of pepsinogen II according to an aspect of the present invention may be a preparation used in one or more tests selected from the group consisting of bacterial agglutination, complement fixation, indirect immunofluorescence test, and immunoassay, specifically, the immunoassay may be one or more selected from the group consisting of enzyme-linked immunosorbent assay (ELISA), chemiluminescent immunoassay, and radioimmunoassay, and more specifically, the enzyme-linked immunosorbent assay may be a latex-enhanced turbidimetric immunoassay, but the type of the preparation is not limited as long as the preparation can measure the level of pepsinogen II in a subject. However, since the unit may be different for each test, the standard of the serum pepsinogen II level is 20 μg/L in the latex-enhanced turbidimetric immunoassay (L-TIA; HBi Corp, Seoul, Korea, imported from Shima Laboratories, Tokyo, Japan), and the standard may be different in the case of other tests.

The subject according to an aspect of the present invention may be under the age of 40. The incidence of gastric cancer is remarkably low in patients under the age of 40, and the Korean National Cancer Screening Program for gastric cancer provides free endoscopy only to those over the age of 40, making it difficult to diagnose the disease at the early-stage stage since patients with diffuse-type gastric cancer under the age of 40 do not have symptoms at the early-stage stage of the disease. According to an embodiment of the present invention, the level of serum pepsinogen II (sPGII) in subjects under the age of 40 have much higher sensitivity and specificity than in subjects over the age of 40 (AUC 0.766, sensitivity 75.0%, specificity 74.2%) (Example 7, and FIGS. 3C and 3D), and from this, it can be seen that the composition according to an aspect of the present invention has an excellent effect of predicting or diagnosing early-stage diffuse-type gastric cancer with high diagnostic power in a sample of a subject under the age of 40.

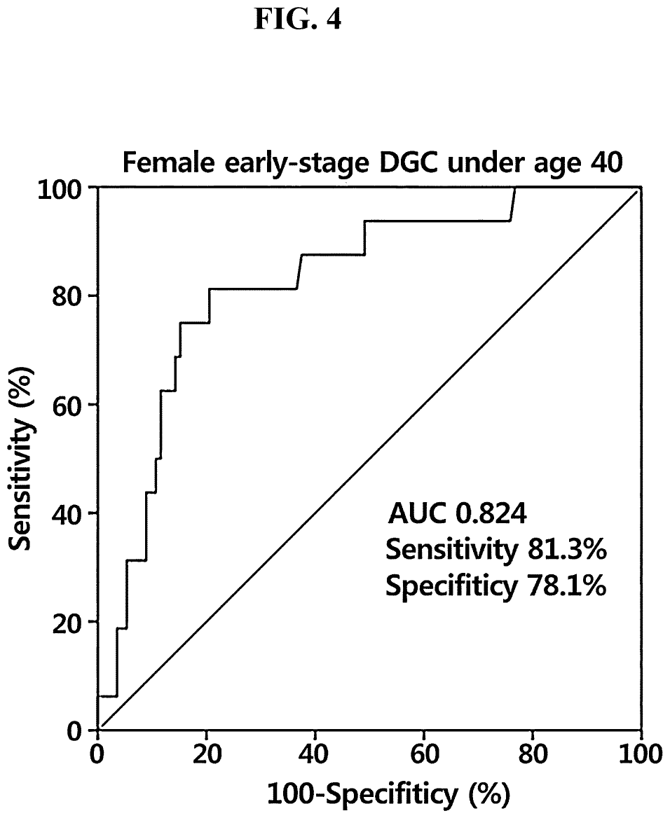

Alternatively, specifically, the subject according to an aspect of the present invention may be a female under the age of 40. Like the previously known epidemiologic features of diffuse-type gastric cancer [Bedikian A Y, Khankhanian N, Heilbrun L K, Bodey G P, Stroehlein J R, Valdivieso M. Gastric carcinoma in young adults. South Med J 1979; 72:654-656.][Isobe T, Hashimoto K, Kizaki J, et al. Characteristics and prognosis of gastric cancer in young patients. Oncol Rep 2013; 30:43-49.41], the risk of diffuse-type gastric cancer is higher in females under the age of 40 than in females over the age of 40, and the rate of diffuse-type gastric cancer is higher in females than in males. According to an embodiment of the present invention, the level of serum pepsinogen II (sPGII) in female subjects under the age of 40 have much higher sensitivity and specificity than in subjects over the age of 40 and subjects including both male and female subjects under the age of 40 (AUC 0.824, sensitivity 81.3%, specificity 78.1%) (Example 7 and FIG. 4), and from this, it can be seen that the composition according to an aspect of the present invention has an excellent effect of predicting or diagnosing early-stage diffuse-type gastric cancer with high diagnostic power in a sample of a female subject under the age of 40. It has been confirmed that whether the level of serum pepsinogen II is equal to or greater than 20 μg/L is a significant biomarker that can determine whether a female subject under the age of 40 has early-stage diffuse-type gastric cancer (Example 8 and Table 4). From this, it can be seen that the composition according to an aspect of the present invention can predict or diagnose early-stage diffuse-type gastric cancer with high diagnostic power in a sample of a female subject under the age of 40, and has an excellent effect in that a subject who is predicted or diagnosed to have early-stage diffuse-type gastric cancer through a test using the composition before endoscopy can be subjected to careful endoscopic follow-up.

The composition according to an aspect of the present invention may be a composition for predicting or diagnosing early-stage diffuse-type gastric cancer. Diffuse-type gastric cancer accounts for about 40% of all gastric cancer cases, but is developed at a relatively young age, has a poorer prognosis than lung cancer, and is usually diagnosed in an advanced state, so the timing of surgery is missed, making it difficult to treat the disease. Therefore, there is a need to discover biomarkers for predicting or diagnosing early-stage diffuse-type gastric cancer. Serum pepsinogen I and the ratio (sPGI/II) of serum pepsinogen I to serum pepsinogen II have been mainly used as pepsinogen as gastric cancer biomarkers in the past, but according to an embodiment of the present invention, the level of serum pepsinogen I and the ratio (sPGI/II) of serum pepsinogen I to serum pepsinogen II are not appropriate as biomarkers for predicting or diagnosing diffuse-type gastric cancer (Example 6 and Table 3). Meanwhile, according to an embodiment of the present invention, among subjects with diffuse-type gastric cancer, 45.5% of subjects have early-stage gastric cancer (EGC), and the proportion of early-stage diffuse-type gastric cancer cases among diffuse-type gastric cancer cases is quite low considering that 73.9% of subjects with intestinal-type gastric cancer have early-stage gastric cancer. A possible mechanism for the onset of diffuse-type gastric cancer is considered to be that Helicobacter pylori-induced inflammation causes several genetic changes in the gastric mucosa [Nardone G, Rocco A, Malfertheiner P. Review article: Helicobacter pylori and molecular events in precancerous gastric lesions. Aliment Pharmacol Ther 2004; 20:261-270][Correa P. Does Helicobacter pylori cause gastric cancer via oxidative stress? Biol Chem 2006; 387:361-364], for example, Helicobacter pylori infection induces CpG island methylation and E-cadherin gene inactivation by DNA methylation [Kang G H, Lee S, Kim J S, Jung H Y. Profile of aberrant CpG island methylation along the multistep pathway of gastric carcinogenesis. Lab Invest 2003; 83:635-641][Chan A O, Lam S K, Wong B C, et al. Promoter methylation of E-cadherin gene in gastric mucosa associated with Helicobacter pylori infection and in gastric cancer. Gut 2003; 52:502-506.][Maekita T, Nakazawa K, Mihara M, et al. High levels of aberrant DNA methylation in Helicobacter pylori-infected gastric mucosae and its possible association with gastric cancer risk. Clin Cancer Res 2006; 12:989-995.][Tamura G, Yin J, Wang S, et al. E-cadherin gene promoter hypermethylation in primary human gastric carcinomas. J Natl Cancer Inst 2000; 92:569-573]. Helicobacter pylori-induced cytotoxin may produce carcinogens such as oxygen free radicals and superoxides, causing mutations in the pepsinogen gene, and these mutations may affect the balance between cell proliferation and differentiation and apoptosis, resulting in diffuse-type gastric cancer [Kuwahara Y, Kono S, Eguchi H, Hamada H, Shinchi K, Imanishi K. Relationship between serologically diagnosed chronic atrophic gastritis, Helicobacter pylori, and environmental factors in Japanese men. Scand J Gastroenterol 2000; 35:476-481.][Backert S, Schwarz T, Miehlke S, et al. Functional analysis of the cag pathogenicity island in Helicobacter pylori isolates from patients with gastritis, peptic ulcer, and gastric cancer. Infect Immun 2004; 72:1043-1056.][Kuniyasu H, Kitadai Y, Mieno H, Yasui W. Helicobactor pylori infection is closely associated with telomere reduction in gastric mucosa. Oncology 2003; 65:275-1056]. According to an embodiment of the present invention, it has been confirmed that the level of serum pepsinogen II is a significant biomarker that can determine whether it is early-stage diffuse-type gastric cancer rather than all gastric cancer cases or all diffuse-type gastric cancer cases (Example 7 and FIG. 3). From this, it can be seen that the composition according to an aspect of the present invention can predict or diagnose early-stage diffuse-type gastric cancer with high diagnostic power in a sample of a subject, and has an excellent effect in that a subject who is predicted or diagnosed to have early-stage diffuse-type gastric cancer through a test using the composition before endoscopy can be subjected to careful endoscopic follow-up.

The sample according to an aspect of the present invention may be one or more selected from the group consisting of serum, plasma, whole blood, urine, saliva and tears, and may specifically be serum.

The composition according to an aspect of the present invention may further contain a preparation for detecting Helicobacter pylori, and the preparation for detecting Helicobacter pylori may be a preparation for one or more tests selected from the group consisting of a histological test, a culture test, a rapid urease test, and a serological test. Specifically, the histological test may be a staining test on a biopsy specimen isolated from the stomach of the subject, the culture test may be a culture test of Helicobacter pylori on a biopsy specimen isolated from the stomach of the subject, and the serological test may be to measure immunoglobulin G (IgG), an antibody against Helicobacter pylori, in the serum of the subject.

More specifically, the immunoassay may be one or more selected from the group consisting of enzyme-linked immunosorbent assay (ELISA), chemiluminescent immunoassay and radioimmunoassay, but is not limited thereto. Helicobacter pylori is known to induce chronic inflammation in the gastric mucosa, leading to chronic active gastritis, atrophy, intestinal metastasis, dysplasia, and gastric cancer in this order. Such a chain reaction is regarded as a major process in gastric carcinogenesis, particularly intestinal-type gastric cancer [Nardone G, Rocco A, Malfertheiner P. Review article: Helicobacter pylori and molecular events in precancerous gastric lesions. Aliment Pharmacol Ther 2004; 20:261-270.][Correa P. Human gastric carcinogenesis: a multistep and multifactorial process—First American Cancer Society Award Lecture on Cancer Epidemiology and Prevention. Cancer Res 1992; 52:6735-6740.][Correa P, Houghton J. Carcinogenesis of Helicobacter pylori. Gastroenterology 2007; 133:659-672.]. However, 20% to 30% of all gastric cancer cases in Western countries occur in the non-atrophic mucosa, and the background gastric mucosa does not show extensive atrophy in 20% to 40% of gastric cancer cases in a Japanese study [Nardone G, Rocco A, Malfertheiner P. Review article: Helicobacter pylori and molecular events in precancerous gastric lesions. Aliment Pharmacol Ther 2004; 20:261-270.][Vauhkonen M, Vauhkonen H, Sipponen P. Pathology and molecular biology of gastric cancer. Best Pract Res Clin Gastroenterol 2006; 20:651-674.][Yanaoka K, Oka M, Mukoubayashi C, et al. Cancer high-risk subjects identified by serum pepsinogen tests: outcomes after 10-year follow-up in asymptomatic middle-aged males. Cancer Epidemiol Biomarkers Prev 2008; 17:838-845.][Ohata H, Oka M, Yanaoka K, et al. Gastric cancer screening of a high-risk population in Japan using serum pepsinogen and barium digital radiography. Cancer Sci 2005; 96:713-720.].

According to an embodiment of the present invention, it is consistent with the previous hypothesis that active inflammation induced by Helicobacter pylori directly induces diffuse-type gastric cancer without undergoing Correa's chain reaction [Sipponen P, Kosunen T U, Valle J, Riihela M, Seppala K. Helicobacter pylori infection and chronic gastritis in gastric cancer. J Clin Pathol 1992; 45:319-323.][Nardone G, Rocco A, Malfertheiner P. Review article: Helicobacter pylori and molecular events in precancerous gastric lesions. Aliment Pharmacol Ther 2004; 20:261-270.][Correa P. Precursors of gastric and esophageal cancer. Cancer 1982; 50:2554-2565.][Laurén P. Histogenesis of intestinal and diffuse types of gastric carcinoma. Scand J Gastroenterol Suppl 1991; 180:160-164.].

According to an embodiment of the present invention, past or current Helicobacter pylori infection may be used as a biomarker for predicting or diagnosing early-stage diffuse-type gastric cancer of a subject, and the risk of early-stage diffuse-type gastric cancer in a subject may be determined when the pepsinogen II level and Helicobacter pylori infection of a subject are used together as biomarkers. Specifically, the preparation for measuring the level of pepsinogen II and the preparation for detecting Helicobacter pylori use the same serum of a subject as the sample for measurement or detection, thus the process is simplified by simultaneously measuring or detecting the two biomarkers using one sample, and there is an excellent effect of predicting or diagnosing early-stage diffuse-type gastric cancer of a subject in a time- and cost-efficient manner through a single-step procedure.

More specifically, the Helicobacter pylori (HP) status of a subject is classified as a positive or negative status based on the following criteria: i) the Helicobacter pylori status is positive if one or more selected from the group consisting of the histological test, culture test, rapid urease test, and serological test are positive; ii) the Helicobacter pylori status is positive if the histological test, culture test, and rapid urease test are all negative and the serological test is positive; iii) the Helicobacter pylori status is positive if the histological test, culture test, rapid urease test, and serological test are negative and the subject has a history of eradication of Helicobacter pylori; and iv) the Helicobacter pylori status is negative if the histological test, culture test, rapid urease test and serological test are negative and the subject does not have a history of eradication of Helicobacter pylori.

Depending on the Helicobacter pylori status together with the level of pepsinogen II in a subject, the subject may be determined as a high-risk group, an intermediate-risk group, or a low-risk group for early-stage diffuse-type gastric cancer based on the following criteria: a) a high-risk group for early-stage diffuse-type gastric cancer if the detected amount of pepsinogen II is equal to or greater than 20 μg/L and the Helicobacter pylori status is positive; b) an intermediate-risk group for early-stage diffuse-type gastric cancer if the detected amount of pepsinogen II is equal to or greater than 20 μg/L or the Helicobacter pylori status is positive; and c) a low-risk group for early-stage diffuse-type gastric cancer if the detected amount of pepsinogen II is less than 20 μg/L and the Helicobacter pylori status is negative.

The subject according to an embodiment of the present invention may be a subject who does not have atrophic gastritis.

In another aspect, the present invention provides a composition for predicting or diagnosing early-stage diffuse-type gastric cancer, comprising a preparation for measuring the level of pepsinogen II (PGII) in the serum of a female under the age of 40 and a preparation for detecting Helicobacter pylori. The subject, age of less than 40, female, serum, preparation for measuring the level of pepsinogen II, preparation for detecting Helicobacter pylori, and early-stage diffuse-type gastric cancer are as described above.

The composition according to an aspect of the present invention has an excellent effect in that the preparation for measuring the level of pepsinogen II and the preparation for detecting Helicobacter pylori use the same serum of a subject as the sample for measurement or detection, the process is simplified by simultaneously measuring or detecting the two biomarkers using one sample, and early-stage diffuse-type gastric cancer of a subject can be predicted or diagnosed in a time- and cost-efficient manner through a single-step procedure.

In still another aspect, the present invention provides a kit for predicting or diagnosing early-stage diffuse-type gastric cancer, comprising the composition for predicting or diagnosing early-stage diffuse-type gastric cancer. The subject, age of less than 40, serum, preparation for measuring the level of pepsinogen II, preparation for detecting Helicobacter pylori, and early-stage diffuse-type gastric cancer are as described above.

The kit according to an aspect of the present invention may be applied to the serum of a subject.

The kit according to an aspect of the present invention may predict or diagnose the subject to have early-stage diffuse-type gastric cancer when the detected amount of pepsinogen II acquired from the sample of a subject is equal to or greater than 20 μg/L.

As an example, the kit according to an aspect of the present invention further includes instructions or an information provision unit, and the instructions or information provision unit may describe or provide information in that a subject is predicted or diagnosed to have early-stage diffuse-type gastric cancer if the detected amount of pepsinogen II acquired from the sample of the subject is equal to or greater than 20 μg/L.

For reference, in the present specification including this paragraph and subsequent paragraphs, the term “unit” of the information provision unit may refer to hardware as well as a combination of software driven by the corresponding hardware. For example, the hardware may be a data processing device including a CPU or other processors. The software driven by hardware may be a program such as a running process, an object, an executable file, a thread of execution, or a calculation program, but is not limited thereto.

According to an embodiment of the present invention, the optimal cutoff value of the serum pepsinogen II (sPGII) level for the prediction or diagnosis of early-stage diffuse-type gastric cancer with a sensitivity of 62.9% and a specificity of 61.2% according to the receiver operating characteristic curve (ROC curve) is 20 μg/L (AUC 0.636) (Example 7 and FIG. 3). The risk of early-stage diffuse-type gastric cancer significantly increases when the serum pepsinogen II (sPGII) level is equal to or greater than 20 μg/L (OR, 3.12) (Example 8 and Table 4), and thus the kit according to an aspect of the present invention has an excellent effect of predicting or diagnosing early-stage diffuse-type gastric cancer with high sensitivity and specificity when the pepsinogen II level of a subject is equal to or greater than 20 μg/L.

The kit according to an aspect of the present invention may include a composition containing a preparation for detecting Helicobacter pylori, the preparation for detecting Helicobacter pylori may be a preparation for one or more tests selected from the group consisting of a histological test, a culture test, a rapid urease test, and a serological test, the histological test may be a staining test on a biopsy specimen isolated from the stomach of the subject, the culture test may be a culture test of Helicobacter pylori on a biopsy specimen isolated from the stomach of the subject, and the serological test may be to measure immunoglobulin G (IgG), an antibody against Helicobacter pylori, in the serum of the subject. Specifically, the serological test may be one or more selected from the group consisting of bacterial agglutination, complement fixation, indirect immunofluorescence test, and immunoassay, and more specifically, the immunoassay may be one or more selected from the group consisting of enzyme-linked immunosorbent assay (ELISA), chemiluminescent immunoassay, and radioimmunoassay, but the type of the preparation is not limited as long as the preparation can detect Helicobacter pylori. The preparation for detecting Helicobacter pylori is as described above.

The kit according to an aspect of the present invention can classify the Helicobacter pylori (HP) status of a subject as a positive or negative status based on the following criteria: i) the Helicobacter pylori status is positive if one or more selected from the group consisting of the histological test, culture test, rapid urease test, and serological test are positive; ii) the Helicobacter pylori status is positive if the histological test, culture test and rapid urease test are all negative and the serological test is positive; iii) the Helicobacter pylori status is positive if the histological test, culture test, rapid urease test and serological test are negative and the subject has a history of eradication of Helicobacter pylori; and iv) the Helicobacter pylori status is negative if the histological test, culture test, rapid urease test and serological test are negative and the subject does not have a history of eradication of Helicobacter pylori.

The criteria to classify the Helicobacter pylori (HP) status of a subject as a positive or negative status described above may be provided by, for example, instructions or an information provision unit in the kit.

The kit according to an aspect of the present invention can classify a subject as a high-risk group, an intermediate-risk group, or a low-risk group for early-stage diffuse-type gastric cancer based on the following criteria: a) a high-risk group for early-stage diffuse-type gastric cancer if the detected amount of pepsinogen II is equal to or greater than 20 μg/L and the Helicobacter pylori status is positive; b) an intermediate-risk group for early-stage diffuse-type gastric cancer if the detected amount of pepsinogen II is equal to or greater than 20 μg/L or the Helicobacter pylori status is positive; and c) a low-risk group for early-stage diffuse-type gastric cancer if the detected amount of pepsinogen II is less than 20 μg/L and the Helicobacter pylori status is negative.

The criteria to classify a subject as a high-risk group, an intermediate-risk group, or a low-risk group for early-stage diffuse-type gastric cancer described above may also be provided by, for example, instructions or an information provision unit in the kit.

According to an embodiment of the present invention, it has been confirmed that the risk of early-stage diffuse-type gastric cancer significantly increases when the Helicobacter pylori (HP) status is positive (OR, 3.03) and that the odds ratio (OR) is 12.76 when the two conditions, the level of serum pepsinogen II and the Helicobacter pylori status, are present together (Example 8 and Table 4), and thus the kit according to an aspect of the present invention has an excellent effect of predicting or diagnosing the risk of early-stage diffuse-type gastric cancer in a subject through the criteria of whether the pepsinogen II level of a subject is equal to or greater than 20 μg/L and whether the Helicobacter pylori status is positive or negative.

In still another aspect, the present invention provides an information provision method for predicting or diagnosing early-stage diffuse-type gastric cancer, comprising measuring the level of pepsinogen II in a sample of a subject under the age of 40. The age of less than 40, sample, pepsinogen II, and early-stage diffuse-type gastric cancer are as described above.

The subject according to an aspect of the present invention may be a female. According to an embodiment of the present invention, the level of serum pepsinogen II (sPGII) in female subjects under the age of 40 have much higher sensitivity and specificity than in subjects over the age of 40 and subjects including both male and female subjects under the age of 40 (AUC 0.824, sensitivity 81.3%, specificity 78.1%) (Example 7 and FIG. 4), and from this, it can be seen that the composition according to an aspect of the present invention has an excellent effect of predicting or diagnosing early-stage diffuse-type gastric cancer with high diagnostic power in a sample of a female subject under the age of 40. It has been confirmed that whether the level of serum pepsinogen II is equal to or greater than 20 μg/L is a significant biomarker that can determine whether a female subject under the age of 40 has early-stage diffuse-type gastric cancer (Example 8 and Table 4). From this, it can be seen that the information provision method according to an aspect of the present invention can predict or diagnose early-stage diffuse-type gastric cancer with high diagnostic power in a sample of a female subject under the age of 40, and has an excellent effect in that a subject who is predicted or diagnosed to have early-stage diffuse-type gastric cancer through a test using the composition before endoscopy can be subjected to careful endoscopic follow-up.

The sample according to an aspect of the present invention may be serum.

The information provision method according to an aspect of the present invention may predict or diagnose the subject to have early-stage diffuse-type gastric cancer if the level of pepsinogen II is equal to or greater than 20 μg/L. According to an embodiment of the present invention, the optimal cutoff value of the serum pepsinogen II (sPGII) level for the prediction or diagnosis of early-stage diffuse-type gastric cancer with a sensitivity of 62.9% and a specificity of 61.2% according to the receiver operating characteristic curve (ROC curve) is 20 μg/L (AUC 0.636) (Example 7 and FIG. 3). The risk of early-stage diffuse-type gastric cancer significantly increases when the serum pepsinogen II (sPGII) level is equal to or greater than 20 μg/L (OR, 3.12) (Example 8 and Table 4), and thus the information provision method according to an aspect of the present invention has an excellent effect of predicting or diagnosing early-stage diffuse-type gastric cancer with high sensitivity and specificity when the pepsinogen II level of a subject is equal to or greater than 20 μg/L.

The information provision method according to an aspect of the present invention may further include measuring whether the subject is infected with Helicobacter pylori (HP), and the measurement of whether the subject is infected with Helicobacter pylori, the preparation for detecting Helicobacter pylori, may include one or more selected from the group consisting of a histological test, a culture test, a rapid urease test, and a serological test. Specifically, the histological test may be a staining test on a biopsy specimen isolated from the stomach of the subject, the culture test may be a culture test of Helicobacter pylori on a biopsy specimen isolated from the stomach of the subject, and the serological test may be to measure immunoglobulin G (IgG), an antibody against Helicobacter pylori, in the serum of the subject. The measurement of whether the subject is infected with Helicobacter pylori is as described above.

The information provision method according to an aspect of the present invention can classify the Helicobacter pylori (HP) status of a subject as a positive or negative status based on the following criteria: i) the Helicobacter pylori status is positive if one or more selected from the group consisting of the histological test, culture test, rapid urease test, and serological test are positive; ii) the Helicobacter pylori status is positive if the histological test, culture test and rapid urease test are all negative and the serological test is positive; iii) the Helicobacter pylori status is positive if the histological test, culture test, rapid urease test and serological test are negative and the subject has a history of eradication of Helicobacter pylori; and iv) the Helicobacter pylori status is negative if the histological test, culture test, rapid urease test and serological test are negative and the subject does not have a history of eradication of Helicobacter pylori. Depending on the level of pepsinogen II and the Helicobacter pylori status of a subject, the information provision method can predict or diagnose the subject to belong to any one of a high-risk group, an intermediate-risk group, or a low-risk group for early-stage diffuse-type gastric cancer: a) a high-risk group for early-stage diffuse-type gastric cancer if the detected amount of pepsinogen II is equal to or greater than 20 μg/L and the Helicobacter pylori status is positive; b) an intermediate-risk group for early-stage diffuse-type gastric cancer if the detected amount of pepsinogen II is equal to or greater than 20 μg/L or the Helicobacter pylori status is positive; and c) a low-risk group for early-stage diffuse-type gastric cancer if the detected amount of pepsinogen II is less than 20 μg/L and the Helicobacter pylori status is negative. According to an embodiment of the present invention, it has been confirmed that the optimal cutoff value of the serum pepsinogen II (sPGII) level for the prediction or diagnosis of early-stage diffuse-type gastric cancer with a sensitivity of 62.9% and a specificity of 61.2% according to the receiver operating characteristic curve (ROC curve) is 20 μg/L (AUC 0.636) (Example 7 and FIG. 3). The risk of early-stage diffuse-type gastric cancer significantly increases when the serum pepsinogen II (sPGII) level is equal to or greater than 20 μg/L (OR, 3.12) and when the Helicobacter pylori (HP) status is positive (OR, 3.03), and the odds ratio (OR) is 12.76 when the two conditions, the level of serum pepsinogen II and the Helicobacter pylori status, are present together (Example 8 and Table 4), and thus the information provision method according to an aspect of the present invention has an excellent effect of predicting or diagnosing the risk of early-stage diffuse-type gastric cancer in a subject through the criteria of whether the pepsinogen II level of a subject is equal to or greater than 20 μg/L and whether the Helicobacter pylori status is positive or negative.

From another point of view, the present invention may relate to a method for predicting or diagnosing early-stage diffuse-type gastric cancer, comprising measuring the level of pepsinogen II (PGII) in a subject under the age of 40 who are in need of prediction or diagnosis of early-stage diffuse-type gastric cancer.

From still another point of view, the present invention may relate to a non-invasive method for predicting or diagnosing early-stage diffuse-type gastric cancer, comprising measuring the level of pepsinogen II (PGII) in a subject under the age of 40 who are in need of prediction or diagnosis of early-stage diffuse-type gastric cancer.

From still another point of view, the present invention may relate to a method for predicting or diagnosing early-stage diffuse-type gastric cancer, comprising measuring the level of pepsinogen II (PGII) and detecting Helicobacter pylori in a subject under the age of 40 who are in need of prediction or diagnosis of early-stage diffuse-type gastric cancer.

From still another point of view, the present invention may relate to a non-invasive method for predicting or diagnosing early-stage diffuse-type gastric cancer, comprising measuring the level of pepsinogen II (PGII) and detecting Helicobacter pylori in a subject under the age of 40 who are in need of prediction or diagnosis of early-stage diffuse-type gastric cancer.

### EXAMPLES

### [Example 1] Subject Enrollment and Examination for Gastric Cancer Biomarker Discovery

In order to discover gastric cancer biomarkers according to an aspect of the present invention, subjects were recruited in the following manner, and the levels of Pepsinogen II and Helicobacter pylori status in each subject were examined.

### [Example 1-1] Subject Enrollment

From February 2006 to March 2017, 2,940 people between the ages of 25 and 80 who visited Seoul National University Bundang Hospital were enrolled as subjects. Among these, 1,124 patients were diagnosed to have gastric cancer (GC) and 353 were diagnosed to have gastric dysplasia by histological analysis. In all, 1,463 subjects with no previous history of gastrointestinal surgery or other malignancies were enrolled as healthy controls. The pathological records of gastric cancer patients who underwent surgery or endoscopic submucosal dissection were reviewed in detail. Gastric cancer was classified according to the Lauren classification and was defined as early-stage gastric cancer if it did not invade more deeply into the submucosa regardless of lymph node metastasis (T1, any N) [Lauren P. The two histological main types of gastric carcinoma: diffuse and so-called intestinal-type carcinoma. an attempt at a histo-clinical classification. Acta Pathol Microbiol Scand 1965; 64:31-49]. It was difficult to classify 30 gastric cancer cases as intestinal-type or diffuse-type gastric cancer. All control subjects with dyspepsia symptoms were examined through gastric endoscopy to exclude other local gastric diseases including gastric mucosa-associated lymphoid tissue lymphoma, gastrointestinal stromal tumor, carcinoid tumor, malignant lymphoma, and esophageal cancer. The Helicobacter pylori (HP) test was also conducted. Subjects with benign diseases such as fundic gland hyperplasia, gastric hyperplastic polyp, mild gastritis, reflux esophagitis, or nonerosive reflux disease were classified as controls. An experienced operator assisted patients in filling out questionnaires and collected blood samples on the day of endoscopy. The study protocol was approved by the Seoul National University Bundang Hospital Clinical Trial Review Board (IRB number: B-1610-368-106) and registered at Clinical trials.gov (NCT03380052). Written informed consent was obtained from all subjects in conformity with the ethical principles of the Declaration of Helsinki.

### [Example 1-2] Measurement of Serum Pepsinogen II (sPGII) Level

The fasting blood samples obtained from the subjects of Example 1-1 were immediately placed in a centrifuge and stored at −70° C. The serum levels of sPGI and sPGII were measured by a latex-enhanced turbidimetric immunoassay (L-TIA; HBi Corp, Seoul, Korea, imported from Shima Laboratories, Tokyo, Japan).

### [Example 1-3] Measurement of Helicobacter pylori (HP) Status

In order to measure the Helicobacter pylori status of the subjects of Example 1-1, ten biopsy specimens were first obtained from the stomach antrum and stomach tissue (corpus) during upper endoscopy for each subject. Using this sample, three diagnostic methods were performed to examine whether the subject is infected with Helicobacter pylori. Specifically, four, which were fixed in 10% neutral-buffered formalin and paraffin-embedded, among the ten biopsy specimens were stained with modified potato solution (Giemsa), hematoxylin and eosin to evaluate the presence of Helicobacter pylori. Four specimens among the remaining specimens were cultured against Helicobacter pylori for 3 to 5 days under microaerobic conditions at 37° C. The last two specimens were subjected to a rapid urease test (Campylobacter-like organism test, CLO test) [Dixon M F, Genta R M, Yardley J H, Correa P. Classification and grading of gastritis. The updated Sydney System. International Workshop on the Histopathology of Gastritis, Houston 1994. Am J Surg Pathol 1996; 20:1161-1181][Loffeld R J, Stobberingh E, Flendrig J A, Arends J W. Helicobacter pylori in gastric biopsy specimens: comparison of culture, modified Giemsa stain, and immunohistochemistry. A retrospective study. J Pathol 1991; 165:69-73][Chey W D, Wong B C; Practice Parameters Committee of the American College of Gastroenterology. American College of Gastroenterology guideline on the management of Helicobacter pylori infection. Am J Gastroenterol 2007; 102:1808-1825].

A serological test of the subjects of Example 1-1 was also performed to measure the Helicobacter pylori status. Specifically, using the blood samples obtained from the subjects of Example 1-2, whether the subject was infected with Helicobacter pylori was tested by an enzyme-linked immunosorbent assay (Genedia HP ELISA; Green Cross Medical Science Corp, Seoul, Yongin) using an immunoglobulin G antibody against Helicobacter pylori with a sensitivity of 97.9% and a specificity of 92.0% in the Korean population [Lee J Y, Kim N, Kim M S, et al. Factors affecting first-line triple therapy of Helicobacter pylori including CYP2C19 genotype and antibiotic resistance. Dig Dis Sci 2014; 59:1235-1243.].

It was defined that the subject was currently and definitely infected with Helicobacter pylori when the subject was positive in one of the histological test, culture test, or rapid urease test (CLO test). In particular, in a case where the subjects were negative in the results of the three Helicobacter pylori tests, histological test, culture test, and rapid urease test, the serological test, namely, anti-HP (Helicobacter pylori) antibody test was used as a qualitative estimation of the history of Helicobacter pylori eradication of all subjects. At this time, it indicates that a subject, who is positive in the serological test for Helicobacter pylori or has a history of Helicobacter pylori eradication, has been infected with Helicobacter pylori in the past.

In conclusion, all subjects were regarded to be in a positive Helicobacter pylori (HP) status if they were infected with Helicobacter pylori currently or in the past, and all subjects were regarded to be in a negative Helicobacter pylori (HP) status if they were not infected with Helicobacter pylori currently or in the past.

### [Example 1-4] Histological Classification of Subject's Stomach

Among the ten biopsy specimens obtained in Examples 1-3, paraffin-embedded specimens were stained with modified potato solution (Giemsa), hematoxylin, and eosin to evaluate the stage of operative link on gastric atrophy (OLGA) and operative link on gastric intestinal metaplasia (OLGIM) of each subject's stomach tissue.

### [Example 2] Statistical Analysis

Using the data on the subjects obtained in Example 1, the baseline characteristics of each group were compared by Student t-test and one-way analysis of variance; and the results were specifically described in Examples 3 to 8 below. The area under the curve (AUC) and receiver operating characteristic curve (ROC curve) were calculated and compared to those of controls to find the optimal cutoff value of sPG for the detection of gastric cancer and gastric dysplasia. It was regarded to be significant when the AUC was 0.7 or more and the sensitivity and specificity were 70% or more. Thereafter, serum pepsinogen I (sPGI), serum pepsinogen II (sPGII) and sPGI/II ratio were divided into two categories. Multivariate logistic regression was used to calculate odds ratios (ORs) of 95% confidence intervals (Cis). Analysis was performed using SPSS version 22.0 (IBM Corp., Armonk, NY, USA). The results were regarded to be significant when the p-value was 0.05 or less.

### [Example 3] Baseline Characteristics of Subjects

The baseline characteristics of the subjects obtained from Example 1 are as shown in Table 1 below.

TABLE 1
Baseline characteristics of control subjects, subjects
with gastric dysplasia and subjects with gastric cancer
Baseline characteristics of control subjects, subjects with
gastric dysplasia and subjects with gastric cancer

| | | | | | | | Diffuse- | |
|---|---|---|---|---|---|---|---|---|
| | | Control | | Gastric | | Intestinal- | type | |
| | Total | subjects | Gastric | cancer | | type gastric | gastric | |
| Character- | (n = | (n = | dysplasia | (n = | | cancer | cancer | |
| istics | 2,940) | 1,463) | (n = 353) | 1,124) | p- Value | (n = 648) | (n = 446) | p- Value |
| Sex | | | | | <0.001* | | | <0.001* |
| Female | 1,235 | 762 | 116 | 357 | | 149 | 198 | |
| | (42.0) | (52.1) | (32.9) | (31.8) | | (23.0) | (44.4) | |
| Male | 1,705 | 701 | 237 | 767 | | 499 | 248 | |
| | (58.0) | (47.9) | (67.1) | (68.2) | | (77.0) | (55.6) | |
| Age, | 56.95 ± | 53.4 ± | 63.63 ± | 59.75 ± | <0.001* | 62.95 ± | 55.1 ± | <0.001* |
| average, | 12.6 | 13.0 | 9.4 | 11.6 | | 9.5 | 12.8 | |
| years old | | | | | | | | |
| <40 | 304 | 240 | 5 | 599 | | 6 | 52 | |
| | (10.3) | (16.4) | (1.4) | (5.20 | | (0.9) | (11.7) | |
| 40-59 | 1,258 | 695 | 119 | 444 | | 207 | 222 | |
| | (42.8) | (47.5) | (33.7) | (39.5) | | (31.9) | (49.8) | |
| ≥60 | 1,378 | 528 | 229 | 621 | | 435 | 172 | |
| | (46.9) | (36.1) | (64.9) | (55.2) | | (67.1) | (38.6) | |
| Smoking | | | | | <0.001* | | | <0.001* |
| Never | 1,411 | 860 | 139 | 412 | | 205 | 196 | |
| | (48.0) | (59.2) | (39.7) | (36.9) | | (31.8) | (44.1) | |
| Ever | 1,510 | 593 | 211 | 706 | | 439 | 248 | |
| | (51.4) | (40.8) | (60.3) | (63.1) | | (68.2) | (55.9) | |
| Drinking | | | | | 0.002* | | | 0.010* |
| Never | 964 | 523 | 1112 | 329 | | 171 | 150 | |
| | (32.8) | (36.0) | (31.9) | (29.5) | | (26.6) | (33.9) | |
| Ever | 1,952 | 928 | 239 | 785 | | 471 | 293 | |
| | (66.4) | (64.0) | (68.1) | (70.5) | | (73.4) | (66.1) | |
| Salty diet | | | | | <0.001* | | | 0.025* |
| Not/ | 2,047 | 1,065 | 247 | 735 | | 408 | 308 | |
| moderate | (69.6) | (76.4) | (72.6) | (68.2) | | (65.6) | (72.1) | |
| Strong | 765 | 329 | 93 | 343 | | 214 | 119 | |
| | (26.0) | (23.6) | (27.4) | (31.8) | | (34.4) | (27.9) | |
| Spicy diet | | | | | 0.012* | | | 0.492 |
| Not/ | 2,028 | 1,033 | 250 | 745 | | 435 | 290 | |
| moderate | (69.0) | (74.9) | (74.2) | (69.6) | | (70.4) | (68.4) | |
| strong | 760 | 347 | 87 | 326 | | 183 | 134 | |
| | (25.9) | (25.1) | (25.8) | (30.4) | | (29.6) | (31.6) | |
| Family | | | | | 0.001* | | | 0.132 |
| history of | | | | | | | | |
| gastic | | | | | | | | |
| cancer | | | | | | | | |
| Negative | 2,205 | 1,049 | 273 | 883 | | 501 | 359 | |
| | (75.0) | (72.5) | (77.6) | (78.9) | | (77.4) | (81.2) | |
| Positive | 711 | 397 (27.5) | 79 | 236 | | 146 | 83 | |
| | (24.2) | | (22.4) | (21.1) | | (22.6) | (18.8) | |
| H. pylori | | | | | <0.001* | | | 0.076 |
| status | | | | | | | | |
| Negative | 580 | 375 | 42 | 163 | | 103 | 54 | |
| | (19.7) | (25.9) | (12.0) | (14.5) | | (15.9) | (12.1) | |
| Positive | 2,338 | 1,072 | 307 | 959 | | 543 | 392 | |
| | (79.5) | (74.1) | (88.0) | (85.5) | | (84.1) | (87.9) | |
| Pepsinogen | | | | | | | | |
| sPGI, μg/L | 62.38 ± | 66.13 ± | 48.84 ± | 61.74 ± | <0.001* | 54.36 ± | 71.52 ± | <0.001* |
| | 49.47 | 52.27 | 42.16 | 47.08 | | 40.37 | 53.82 | |
| sPGII, μg/L | 21.59 ± | 21.23 ± | 17.45 ± | 23.35 ± | <0.001* | 20.82 ± | 26.47 ± | <0.001* |
| | 22.98 | 23.03 | 13.75 | 24.97 | | 21.22 | 27.78 | |
| sPGI/II | 3.66 ± | 4.17 ± | 2.93 ± | 3.21 ± | <0.001* | 3.15 ± | 3.30 ± | 0.242 |
| ratio | 2.73 | 3.24 | 1.63 | 2.05 | | 2.25 | 1.76 | |
| Atrophy† | | | | | <0.001* | | | <0.001* |
| OLGA 0 | 663 | 458 | 35 | 170 | | 70 | 92 | |
| | (22.6) | (53.0) | (18.8) | (25.9) | | (18.2) | (36.9) | |
| OLGA I | 517 | 266 | 46 | 205 | | 120 | 78 | |
| | (17.6) | (30.8) | (24.7) | (31.2) | | (31.3) | (31.3) | |
| OLGA II | 320 | 98 | 49 | 173 | | 123 | 43 | |
| | (10.9) | (11.3) | (26.3) | (26.3) | | (32.0) | (17.3) | |
| OLGA III | 147 | 30 | 35 | 82 | | 52 | 28 | |
| | (5.0) | (3.5) | (18.0) | (12.5) | | (13.5) | (11.2) | |
| OLGA IV | 60 | 12 | 21 | 27 | | 19 | 8 | |
| | (2.0) | (1.4) | (11.3) | (4.1) | | (4.9) | (3.2) | |
| Metaplasia† | | | | | <0.001* | | | <0.001* |
| OLGIM 0 | 1,197 | 865 | 49 | 283 | | 101 | 173 | |
| | (40.7) | (63.3) | (14.1) | (25.7) | | (15.8) | (40.0) | |
| OLGIM I | 600 | 251 | 84 | 265 | | 149 | 107 | |
| | (20.4) | (18.4) | (24.1) | (24.1) | | (23.4) | (24.8) | |
| OLGIM II | 561 | 174 | 95 | 292 | | 199 | 85 | |
| | (19.3) | (12.7) | (27.3) | (26.5) | | (31.2) | (19.7) | |
| OLGIM III | 322 | 59 | 72 | 191 | | 131 | 57 | |
| | (11.0) | (4.3) | (20.7) | (17.4) | | (20.5) | (13.2) | |
| OLGIM IV | 135 | 18 | 48 | 69 | | 58 | 10 | |
| | (4.6) | (1.3) | (13.8) | (6.3) | | (9.1) | (2.3) | |

Data are presented as numbers (%) or median ± SD.
GC, gastric cancer; H. pylori, Helicobacter pylori; sPG, serum pepsinogen; OLGA, operative link on gastric atrophy; OLGIM, operative link on gastric intestinal metaplasia.
*Asterisk indicates statistical significance;
†Subjects excluding specimens inapplicable to atrophy and intestinal metaplasia because of problems such as improper fixation, incorrect orientation, and dense inflammation.

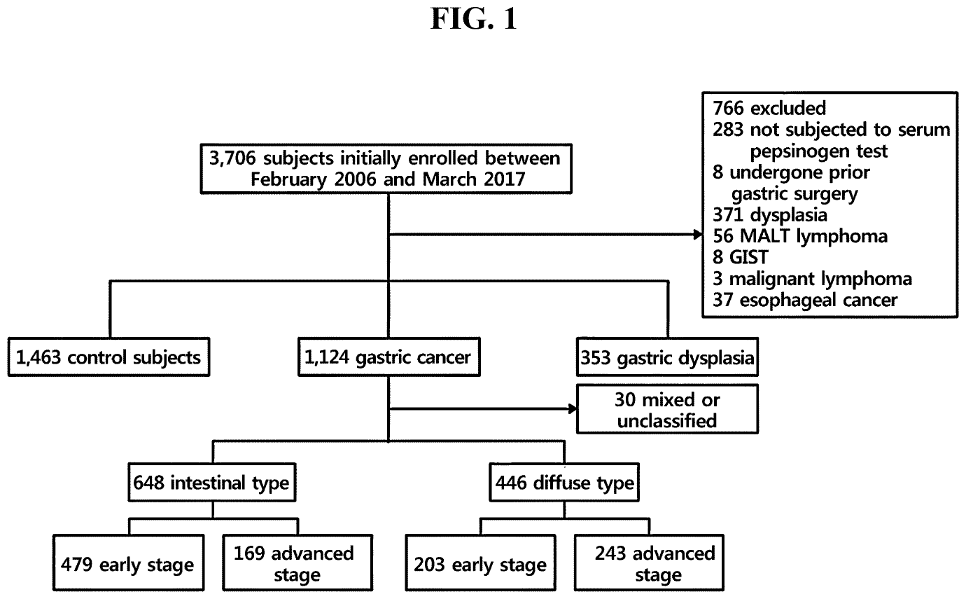

Table 1 shows the baseline characteristics of gastric dysplasia (n=353), gastric cancer (n=1,124) and control group (n=1,463). The average age of the control group was 53.4±13.0 years, which was significantly lower than that of the gastric cancer (59.75±11.6 years) and gastric dysplasia (62.63±9.4 years) groups. According to the summed one-way analysis of variance, there were significant differences in sex, smoking/drinking history, salty/spicy diet, family history, Helicobacter pylori (HP) status, sPGI, sPGII, sPGI/II ratio, OLGA stage and OLGIM stage among the three groups of control group, gastric dysplasia group, and gastric cancer group (Table 1). When gastric cancer was histologically classified into an intestinal type and a diffuse type, it was difficult to classify 30 patients with gastric cancer as an intestinal type or a diffuse type (FIG. 1). Therefore, the remaining 1,094 patients were classified into an intestinal-type gastric cancer (IGC) group and a diffuse-type gastric cancer (DGC) group, among which 648 patients were classified as the intestinal-type gastric cancer (IGC) group (59.2%) and 446 patients as the diffuse-type gastric cancer (DGC) group (40.8%). Differences in sex, smoking/drinking history, salty diet, sPGI and sPGII, OLGA stage, and OLGIM stage were observed in gastric cancer subgroups (Table 1).

### [Example 4] Comparison of Serological and Histological Characteristics Related to Gastric Cancer Stage

The serological and histological characteristics in Examples 1-2 to 1-4 of the subjects belonging to the gastric cancer group in Example 3 were compared, and the results are as shown in Table 2 below.

TABLE 2
Comparison of serological and histological characteristics related to gastric cancer stage
Comparison of serological and histological characteristics related to gastric cancer stage

| | | | | Intestinal-type gastric | | | Diffuse-type gastric | | |
|---|---|---|---|---|---|---|---|---|---|
| | Total | | | cancer | | | cancer | | |
| | EGC | AGC | | EGC | AGC | | EGC | AGC | |
| Variable | (n = 697) | (n = 424) | p- value | (n = 497) | (n = 169) | p- value | (n = 203) | (n = 243) | p- value |
| H. pylori | | | 0.572 | | | 0.797 | | | 0.297 |
| status | | | | | | | | | |
| Negative | 98 | 65 | | 75 | 28 | | 21 | 33 | |
| | (14.1) | (153) | | (15.7) | (16.6) | | (10.3) | (13.6) | |
| Positive | 597 | 359 | | 402 | 141 | | 182 | 210 | |
| | (85.9) | (84.7) | | (84.3) | (83.4) | | (89.7) | (86.4) | |
| Pepsinogen | | | | | | | | | |
| sPGI, | 61.79 ± | 61.75 ± | 0.990 | 53.83 ± | 55.87 ± | 0.571 | 79.71 ± | 64.72 ± | 0.003* |
| μg/L | 43.53 | 52.55 | | 38.75 | 44.73 | | 48.43 | 57.13 | |
| sPGII, | 22.71 ± | 24.48 ± | 0.251 | 19.14 ± | 25.58 ± | 0.001* | 30.76 ± | 22.91 ± | 0.003* |
| μg/L | 34.35 | 27.47 | | 17.78 | 28.36 | | 31.72 | 23.49 | |
| sPGI/II | 3.30 ± | 3.07 ± | 0.069 | 3.29 ± | 2.73 ± | 0.005* | 3.31 ± | 3.29 ± | 0.896 |
| ratio | 2.19 | 2.19 | | 2.40 | 1.68 | | 1.66 | 1.83 | |
| OLGA† | | | | <0.001* | | <0.001* | | | <0.001* |
| Low risk | 355 | 190 | | 242 | 71 | | 102 | 111 | |
| | (85.3) | (79.8) | | (84.9) | (71.7) | | (85.0) | (86.0) | |
| High risk | 61 | 48 | | 43 | 28 | | 18 | 18 | |
| | (14.7) | (20.2) | | (15.1) | (28.3) | | (15.0) | (14.0) | |
| OLGIM† | | | <0.001* | | | <0.001* | | | <0.001* |
| Low risk | 533 | 304 | | 348 | 101 | | 172 | 193 | |
| | (72.4) | (74.5) | | (73.3) | (62.0) | | (86.4) | (82.8) | |
| High risk | 156 | 104 | | 127 | 62 | | 27 | 40 | |
| | (22.6) | (25.5) | | (25.7) | (38.0) | | (13.6) | (17.2) | |

Data are presented as numbers (%) or median ± SD.
GC, gastric cancer; EGC, early gastric cancer; AGC, advanced gastric cancer, H. pylori, Helicobacter pylori; sPG, serum pepsinogen; OLGA, operative link on gastric atrophy; OLGIM, operative link on gastric intestinal metaplasia.
*Asterisk indicates statistical significance;
†Subjects excluding specimens inapplicable to atrophy and intestinal metaplasia because of problems such as improper fixation, incorrect orientation, and dense inflammation.

First, with regard to the Helicobacter pylori (HP) status and serum pepsinogens (sPGs), statistically significant differences were not observed between the early-stage GC and advanced-stage GC groups (Table 2). Even when gastric cancer was classified depending on the histological type, there was still no difference in the Helicobacter pylori (HP) status. However, the level of serum pepsinogen II (sPGII) was higher (p=0.001) and the sPGI/II ratio was lower (p=0.005) in the advanced-stage intestinal-type gastric cancer group than in the early-stage intestinal-type gastric cancer group. Patients with early-stage diffuse-type gastric cancer (DGC) had higher levels of serum pepsinogen I (sPGI) (p=0.003) and serum pepsinogen II (sPGII) (p=0.003) than patients with advanced-stage diffuse-type gastric cancer. With regard to the histological characteristics, the advanced-stage intestinal-type gastric cancer group had more advanced atrophic gastritis and intestinal metaplasia than the early-stage intestinal-type cancer group (Table 2).

As shown in Table 2, serum pepsinogen II (sPGII) was higher in subjects in the advanced stage than in the early stage in the case of intestinal-type gastric cancer, unlike diffuse-type gastric cancer. In the study by Stemmermann and Nomura [Stemmermann G N, Nomura A M. The relation of pepsinogen group II (PGII) expression to intestinal metaplasia and gastric cancer. Histopathology 2006; 49:45-51], serum pepsinogen II (sPGII) was more likely to be expressed in patients with moderate or extensive intestinal metaplasia than in subjects with minimal or no intestinal metaplasia. In the above study, cancers that expressed serum pepsinogen II (sPGII) were found to be at a higher stage than cancers that did not, and it was hypothesized that cancers originating in the intestinalized glands could later revert to the gastric phenotype. Accordingly, the present inventors hypothesized that high serum pepsinogen II (sPGII) in advanced-stage intestinal-type gastric cancer could be derived from postinduction reversion after induction from intestinal cells to the gastric phenotype, but it was confirmed that serum pepsinogen II (sPGII) was not so high in this phenomenon in the case of early-stage intestinal-type gastric cancer.

### [Example 5] Relation Between Helicobacter pylori Status and Serum Pepsinogen (sPG) and OLGA/OLGIM Stage

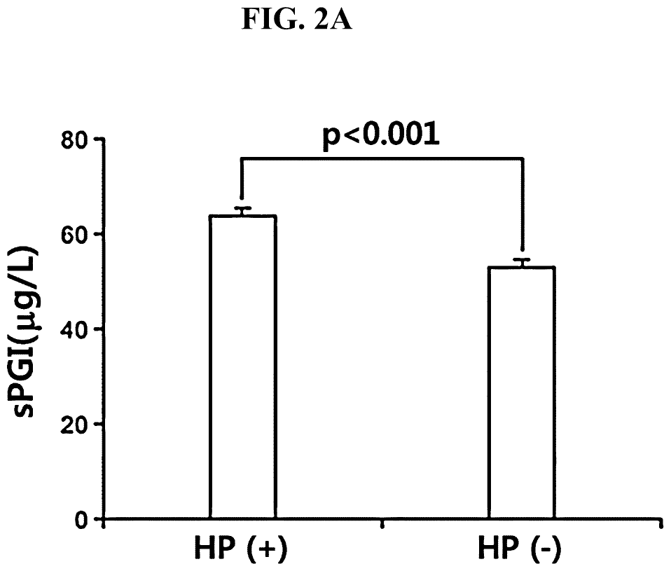

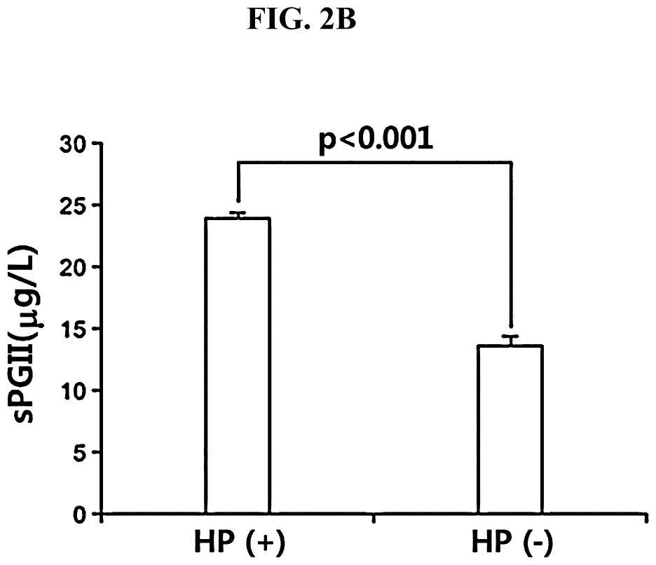

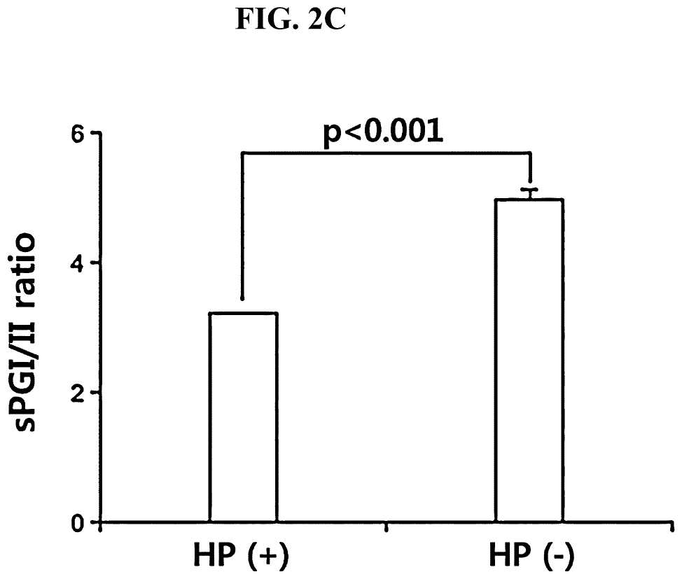

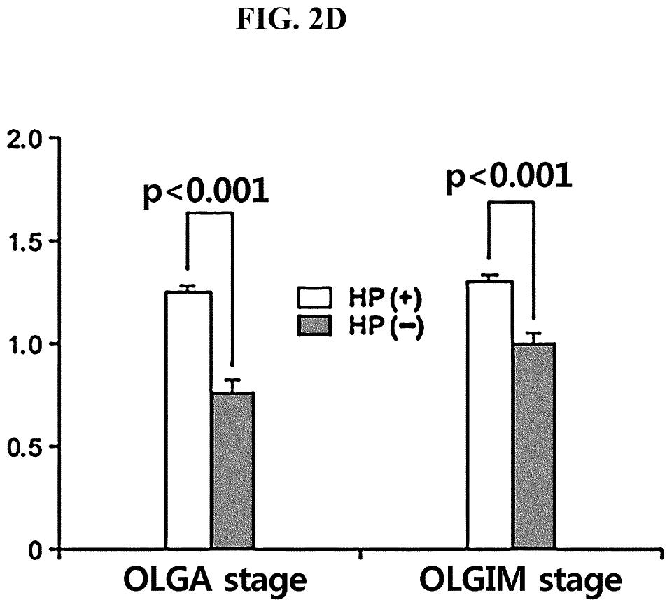

The relation between the levels of serum pepsinogens (sPGs) and the Helicobacter pylori (HP) status in the OLGA/OLGIM stage of the subjects in Examples 1-2 to 1-4 was examined, and the results are as illustrated in FIG. 2.

As illustrated in FIGS. 2A to 2C, there were significant differences in serum pepsinogens (sPGs) depending on the Helicobacter pylori (HP) status of subjects, and subjects with a positive HP status had higher levels of serum pepsinogen I (sPGI) and serum pepsinogen II (sPGII) (FIGS. 2A and 2B) and lower sPGI/II ratios (FIG. 2C) than subjects with a negative HP status (all p<0.001). The OLGA and OLGIM stages were higher in subjects with a positive HP status, an indicator of atrophic gastritis and intestinal metaplasia (p<0.001) (FIG. 2D).

### [Example 6] Correlation Between Serum Pepsinogens (sPGs) and Helicobacter pylori Status and Gastric Dysplasia or Gastric Cancer

The relation between the levels of serum pepsinogens (sPGs) and the Helicobacter pylori (HP) status of the subjects in Examples 1-2 to 1-4 and the risk of gastric dysplasia and gastric cancer is shown in Table 3.

TABLE 3
Correlation between serum pepsinogens (sPGs), HP status, gastric dysplasia, and gastric cancer
Correlation between serum pepsinogens (sPGs), HP status, gastric dysplasia, and gastric cancer

| | | | Intestinal-type gastric | | | | | |
|---|---|---|---|---|---|---|---|---|
| | Gastric | Gastric | cancer | | | Diffuse-type gastric cancer | | |
| Variable | dysplasia | cancer | Total | EGC | AGC | Total | EGC | AGC |
| sPGI, | | | | | | | | |
| μg/L | | | | | | | | |
| ≥70 | 1 | 1 | 7 | 1 | 1 | 1 | 1 | 1 |
| <70 | 2.06 | 1.00 | 1.68 | 1.68 | 1.64 | 0.68 | 0.48 | 0.94 |
| | (<0.001)* | (0.996) | (<0.001)* | (<0.001)* | (0.003)* | (0.001)* | (<0.001)* | (0.680) |
| sPGII, | | | | | | | | |
| μg/L | | | | | | | | |
| <20 | 1 | 1 | 1 | 1 | 1 | 1 | 1 | |
| ≥20 | 0.72 | 1.32 | 1.04 | 0.97 | 1.24 | 1.78 | 3.12 | 1.12 |
| | (0.009)* | (<0.001)* | (0.722) | (0.775) | (0.189) | (<0.001)* | (<0.001)* | (0.426) |
| sPGI/II | | | | | | | | |
| ratio | | | | | | | | |
| ≥3 | 1 | 1 | 1 | 1 | 1 | 1 | 1 | 1 |
| <3 | 2.77 | 2.25 | 2.50 | 2.25 | 3.40 | 1.93 | 1.96 | 1.91 |
| | (<0.001)* | (<0.001)* | (<0.001)* | (<0.001)* | (<0.001)* | (<0.001)* | (<0.001)* | (<0.001)* |
| HP status | | | | | | | | |
| (−) | 1 | 1 | 1 | 1 | 1 | 1 | 1 | 1 |
| (+) | 2.56 | 2.06 | 1.84 | 1.88 | 1.76 | 2.54 | 3.03 | 2.23 |
| | (<0.001)* | (<0.001)* | (<0.001)* | (<0.001)* | (0.009)* | (<0.001)* | (<0.001)* | (<0.001)* |

Data are presented as odds ratios (p-values).
Logistic model adjusted for sex and age.
sPG, serum pepsinogen; HP, Helicobacter pylori, GC, gastric cancer; EGC, early gastric cancer; AGC, advanced gastric cancer.
*Asterisk indicates statistical significance.

As shown in Table 3, the odds ratio (OR) of the group having an sPGI//II ratio of less than 3 was 2.77 in the gastric dysplasia group and 2.25 in the gastric cancer group. Among the subtypes of gastric cancer, the odds ratio of the intestinal-type gastric cancer group (IGC) was higher than that of the diffuse-type gastric cancer group (DGC) when the sPGI/II ratio was less than 3 (OR, 2.50; p<0.001), and was much higher particularly in advanced-stage gastric cancer subjects (OR, 3.40; p<0.001). In contrast, when the level of serum pepsinogen II (sPGII) was equal to or greater than 20 μg/L, the odds ratio was higher in the diffuse-type gastric cancer group (DGC) (OR, 1.78; p<0.001), and was higher particularly in early-stage gastric cancer subjects (OR, 3.12; p<0.001), but was not higher in the intestinal-type gastric cancer group (IGC). A level of serum pepsinogen I (sPGI) of less than 70 μg/L was similar in the gastric dysplasia group (OR, 2.06; p<0.001) and the intestinal-type gastric cancer group (IGC) (OR, 1.68; p<0.001) compared to the sPGI/II ratio, but the overall odds ratio was low. The level of serum pepsinogen I (sPGI) was not associated with all gastric cancer cases; instead, a high level of serum pepsinogen I (sPGI) was associated with diffuse-type gastric cancer (DGC). When the significance of HP status was examined, a positive HP status was associated with increased risk of gastric dysplasia (OR, 2.56; p<0.001) and gastric cancer (OR, 2.06; p<0.001). Among the gastric cancer types, the risk of diffuse-type gastric cancer (DGC) was remarkably higher than the risk of intestinal-type gastric cancer (IGC) when the HP status was positive (OR, 2.54; p<0.001).

Considering that serum pepsinogen I and the ratio (sPGI/II) of serum pepsinogen I to serum pepsinogen II have been mainly used as pepsinogens for gastric cancer biomarkers in the past, the results indicate that the level of serum pepsinogen I and the ratio (sPGI/II) of serum pepsinogen I to serum pepsinogen II are not appropriate as biomarkers for predicting or diagnosing diffuse-type gastric cancer. Specifically, the odds ratio (OR) of serum pepsinogen I level compared to that in the control group for gastric cancer was not significant (OR, 1.00) as a biomarker for gastric cancer screening in Korea. When limiting the gastric cancer to intestinal-type gastric cancer, the odds ratio is 1.68 (1.68 for early-stage gastric cancer and 1.84 for advanced-stage gastric cancer), but pepsinogen is still insufficient to be used as a biomarker for gastric cancer diagnosis when compared to conventional Japanese studies [Yoshihara M, Sumii K, Haruma K, et al. Correlation of ratio of serum pepsinogen I and II with prevalence of gastric cancer and adenoma in Japanese subjects. Am J Gastroenterol 1998; 93:1090-1096.][Miki K. Gastric cancer screening using the serum pepsinogen test method. Gastric Cancer 2006; 9:245-253][Kitahara F, Kobayashi K, Sato T, Kojima Y, Araki T, Fujino M A. Accuracy of screening for gastric cancer using serum pepsinogen concentrations. Gut 1999; 44:693-697.][Dinis-Ribeiro M, Yamaki G, Miki K, Costa-Pereira A, Matsukawa M, Kurihara M. Meta-analysis on the validity of pepsinogen test for gastric carcinoma, dysplasia or chronic atrophic gastritis screening. J Med Screen 2004; 11:141-147.][Hattori Y, Tashiro H, Kawamoto T, Kodama Y Sensitivity and specificity of mass screening for gastric cancer using the measurement of serum pepsinogens. Jpn J Cancer Res 1995; 86:1210-1215]. These results are presumed to be due to various factors such as the difference in the ratio of intestinal-type gastric cancer to diffuse-type gastric cancer, Helicobacter pylori infection rate, Helicobacter pylori eradication rate, and differences in test equipment [Oishi Y, Kiyohara Y, Kubo M, et al. The serum pepsinogen test as a predictor of gastric cancer: the Hisayama study. Am J Epidemiol 2006; 163:629-637][Ohata H, Kitauchi S, Yoshimura N, et al. Progression of chronic atrophic gastritis associated with Helicobacter pylori infection increases risk of gastric cancer. Int J Cancer 2004; 109:138-143.][Watabe H, Mitsushima T, Yamaji Y, et al. Predicting the development of gastric cancer from combining Helicobacter pylori antibodies and serum pepsinogen status: a prospective endoscopic cohort study. Gut 2005; 54:764-768]. A cutoff of less than 3 for the PGI/II ratio has an odds ratio of 2.77 in gastric dysplasia and 2.25 in gastric cancer, but the sPGI/II ratio is more suitable as a biomarker in intestinal-type gastric cancer than in diffuse-type gastric cancer and in advanced-stage gastric cancer rather than in early-stage gastric cancer, and is most appropriate particularly in advanced-stage intestinal-type gastric cancer (OR, 3.40). These results are considered to be associated with different grades of atrophic gastritis and intestinal metaplasia according to the histological type and stage. OLGA and OLGIM stages were actually higher in intestinal-type gastric cancer than in diffuse-type gastric cancer and in advanced-stage gastric cancer than in early-stage gastric cancer, and it has been confirmed that the sPGI/II ratio is an appropriate biomarker for atrophic gastritis and closely associated with intestinal-type gastric cancer.

### [Example 7] Confirmation of Detectability of Serum Pepsinogen II (sPGII) for Prediction or Diagnosis of Gastric Cancer

Through Example 6, it was confirmed that a high level of serum pepsinogen II (sPGII) could be a biomarker for predicting or diagnosing early-stage diffuse-type gastric cancer, and in order to confirm its usefulness, AUC (area under the curve) was calculated and the results are illustrated in FIGS. 3 and 4.

As illustrated in FIGS. 3 and 4, serum pepsinogen II (sPGII) did not have a significant AUC with sensitivity and specificity for all gastric cancer cases (FIG. 3A) and all diffuse-type gastric cancer cases (FIG. 3B). However, when diffuse-type gastric cancer was divided into early-stage and advanced-stage cancers, AUC increased in early-stage diffuse-type gastric cancer. According to the receiver operating characteristic curve (ROC curve), the optimal cutoff value of the serum pepsinogen II (sPGII) level for the prediction or diagnosis of early-stage diffuse-type gastric cancer with a sensitivity of 62.9% and specificity of 61.2% was 20 μg/L (AUC 0.636). When the subjects with early-stage diffuse-type gastric cancer were classified based on the age of 40, the level of serum pepsinogen II (sPGII) had far significantly higher diagnostic power in subjects under the age of 40 than in subjects over the age of 40 (FIGS. 3C and 3D) (AUC 0.766, sensitivity 75.0%, specificity 74.2%). The level of serum pepsinogen II (sPGII) in female subjects under the age of 40 had much higher sensitivity and specificity than in subjects over the age of 40 and subjects including both male and female subjects under the age of 40 (AUC 0.824, sensitivity 81.3%, specificity 78.1%) (FIG. 4).

### [Example 8] Correlation Between Serum Pepsinogen II (sPGII) and Helicobacter pylori (HP) Status for Prediction or Diagnosis of Diffuse-Type Gastric Cancer

In order to find the most powerful model for predicting or diagnosing diffuse-type gastric cancer by collecting the results of Examples 1 to 7 together, risk stratification analysis was performed, and the results are as shown in Table 4.

TABLE 4
Risk stratification of diffuse-type gastric cancer by
combination of serum pepsinogen II (sPGII) and HP status
Risk stratification of diffuse-type gastric cancer by
combination of serum pepsinogen II (sPGII) and HP status

| | | | | Early | Advanced- |
|---|---|---|---|---|---|
| | | | | stage | stage |
| | | | Diffuse- | diffuse- | diffuse- |
| | | | type | type | type |
| | | HP/ | gastric | gastric | gastric |
| Variable | Risk | sPGII | cancer | cancer | cancer |
| Total | Low | −/− | 1 | 1 | 1 |
| | Interme- | +/− | 2.26 | 1.82 | 2.53 |
| | diate | | (<0.001) | (0.040) | (<0.001) |
| | | −/+ | 1.57 | 1.55 | 1.59 |
| | | | (0.193) | (0.411) | (0.292) |
| | High | +/+ | 3.44 | 5.20 | 1.92 |
| | | | (<0.001) | (<0.001) | (0.013) |
| Age ≥40 | Low | −/− | 1 | 1 | 1 |
| years | Interme- | +/− | 2.21 | 1.73 | 2.32 |
| | diate | | (<0.001) | (0.079) | (<0.001) |
| | | −/+ | 1.55 | 1.49 | 1.46 |
| | | | (0.219) | (0.460) | (0.391) |
| | High | +/+ | 2.88 | 4.32 | 1.83 |
| | | | (<0.001) | (<0.001) | (0.021) |
| Age <40 | Low | −/− | 1 | 1 | 1 |
| years | Interme- | +/− | 1.63 | 1.63 | 1.63 |
| | diate | | (0.353) | (0.591) | (0.442) |
| | | −/+ | 0.00 | 0.00 | 0.00 |
| | | | (0.999) | (0.999) | (0.999) |
| | High | +/+ | 8.04 | 12.76 | 5.67 |
| | | | (<0.001) | (0.001) | (0.003) |
| Male <40 | Low | −/− | 1 | 1 | 1 |
| years | Interme- | +/− | 3.38 | 3.12 | 3.64 |
| | diate | | (0.121) | (0.300) | (0.235) |
| | | −/+ | 0.00 | 0.00 | 0.00 |
| | | | (0.999) | (0.999) | (0.999) |
| | High | +/+ | 5.00 | 5.00 | 5.00 |
| | | | (0.133) | (0.272) | (0.282) |
| Female <40 | Low | −/− | 1 | 1 | 1 |
| years | Interme- | +/− | 4.24 | 6.46 | 3.50 |
| | diate | | (0.013) | (0.084) | (0.063) |
| | | −/+ | 0.00 | 0.00 | 0.00 |
| | | | (0.999) | (0.999) | (0.999) |
| | High | +/+ | 0.750 | 21.00 | 3.00 |
| | | | (0.003)* | (0.066)* | (0.542) |

Data are presented as odds ratios (p-values).
Low risk, (HP−/PG−); Intermediate risk, (HP+/PG−) or (HP−/PG+); High risk, (HP+/PG+).
Subjects with PG II ≥20 μg/L are classified as PG (+).
Logistic model adjusted for sex and age.
GC, gastric cancer;
sPG, serum pepsinogen;
HP, helicobacter pylori;
DGC, diffuse gastric cancer
*Asterisk indicates statistical significance.

As shown in Table 4, depending on the Helicobacter pylori (HP) status and the serum pepsinogen II (sPGII) level of 20 μg/L, a subject was classified as the low-risk group if the Helicobacter pylori (HP) status was negative and serum pepsinogen II (sPGII) was less than 20 μg/L, a subject was classified as the intermediate-risk group if the Helicobacter pylori (HP) status was positive or serum pepsinogen II (sPGII) was equal to or greater than 20 μg/L, and a subject was classified as the high-risk group if the Helicobacter pylori (HP) status was positive and serum pepsinogen II (sPGII) was equal to or greater than 20 μg/L. Compared with the low-risk group, the odds ratio of diffuse-type gastric cancer in the high-risk group was 3.44 (p<0.001). The early-stage diffuse-type gastric cancer group (OR, 5.20; p<0.001) had higher association with the high-risk group than the advanced-stage diffuse-type gastric cancer group (OR, 1.92; p=0.013). Meanwhile, when subgroups were analyzed by age and sex of the subjects, in the high-risk group, the odds ratio of early-stage diffuse-type gastric cancer at the age of 40 or less was 12.76 (p=0.001), and the odds ratio of early-stage diffuse-type gastric cancer was the highest in females under the age of 40 (OR, 21.00; p=0.006).

In other words, it has been confirmed that the risk of early-stage diffuse-type gastric cancer is significantly increased when the serum pepsinogen II (sPGII) level is equal to or greater than 20 μg/L (OR, 3.12) and the Helicobacter pylori (HP) status is positive (OR, 3.03), and the odds ratio (OR) is 12.76 when these two conditions are present together.

Overall, according to an embodiment of the present invention, it has been confirmed that a composition comprising a preparation capable of measuring the level of serum pepsinogen II (sPGII) in a subject can predict or diagnose early-stage diffuse-type gastric cancer, and furthermore, early-stage diffuse-type gastric cancer can be predicted or diagnosed with high sensitivity and specificity by the history of current or past infection with Helicobacter pylori (HP) together with the level of serum pepsinogen II. In particular, it has been found that the level of serum pepsinogen II and the Helicobacter pylori (HP) status can be usefully employed as non-invasive markers that may be performed before endoscopy in females under the age of 40 who are at high risk of diffuse-type gastric cancer.

### INDUSTRIAL APPLICABILITY

In an aspect of the present invention, serum pepsinogen II has been discovered as a biomarker for predicting or diagnosing early-stage diffuse-type gastric cancer, thus it is possible to predict or diagnose early-stage diffuse-type gastric cancer with high sensitivity and specificity by measuring serum pepsinogen II as a non-invasive means before endoscopy. Furthermore, the process is simplified by simultaneously measuring or detecting the serum pepsinogen II level and the Helicobacter pylori infection history in the same sample, for example, serum, and serum pepsinogen II can be used to predict or diagnose early-stage diffuse-type gastric cancer of a subject in a time- and cost-effective manner through a single-step procedure.

1. A method for predicting or diagnosing early-stage diffuse-type gastric cancer, comprising measuring a level of pepsinogen II (PGII) in a sample of a subject under age of 40.

2. The method according to claim 1, wherein the subject is a female.

3. The method according to claim 1,
wherein the sample is one or more selected from the group consisting of serum, plasma, whole blood, urine, saliva, and tears.

4. The method according to claim 3,
wherein the sample is serum.

5. The method according to claim 1,
wherein the method further comprises detecting Helicobacter pylori.

6. A method for predicting or diagnosing early-stage diffuse-type gastric cancer, comprising measuring a level of pepsinogen II (PGII) in serum of a female under age of 40 and detecting Helicobacter pylori.

7. A kit for predicting or diagnosing early-stage diffuse-type gastric cancer, comprising a composition comprising measuring a level of pepsinogen II (PGII) in a sample of a subject under age of 40.

8. The kit according to claim 7,
wherein the kit is applied to serum of a subject.

9. The kit according to claim 7,
wherein the kit predicts or diagnoses a subject to have early-stage diffuse-type gastric cancer when a detected amount of pepsinogen II acquired from a sample of the subject is equal to or greater than 20 μg/L.

10. The kit according to claim 7,
wherein the kit comprises a composition containing detecting Helicobacter pylori,
the preparation for detecting Helicobacter pylori is a preparation for one or more tests selected from the group consisting of a histological test, a culture test, a rapid urease test, and a serological test,
the histological test is a staining test on a biopsy specimen isolated from stomach of the subject,
the culture test is a culture test of Helicobacter pylori on a biopsy specimen isolated from stomach of the subject, and
the serological test is to measure immunoglobulin G (IgG), an antibody against Helicobacter pylori, in serum of the subject.

11. The kit according to claim 10,
wherein the serological test is one or more selected from the group consisting of bacterial agglutination, complement fixation, indirect immunofluorescence test, and immunoassay.

12. The kit according to claim 11,
wherein the immunoassay is one or more selected from the group consisting of enzyme-linked immunosorbent assay (ELISA), chemiluminescent immunoassay, and radioimmunoassay.

13. The kit according to claim 10,
wherein the kit has the following criteria to classify a Helicobacter pylori (HP) status of a subject as a positive or negative status:
i) the Helicobacter pylori status is positive if one or more selected from the group consisting of the histological test, culture test, rapid urease test, and serological test are positive;
ii) the Helicobacter pylori status is positive if the histological test, culture test, and rapid urease test are all negative and the serological test is positive;
iii) the Helicobacter pylori status is positive if the histological test, culture test, rapid urease test, and serological test are negative and the subject has a history of eradication of Helicobacter pylori; and
iv) the Helicobacter pylori status is negative if the histological test, culture test, rapid urease test, and serological test are negative and the subject does not have a history of eradication of Helicobacter pylori.

14. The kit according to claim 13,
wherein the kit has the following criteria to classify a subject as a high-risk group, an intermediate-risk group, or a low-risk group for early-stage diffuse-type gastric cancer:
a) a high-risk group for early-stage diffuse-type gastric cancer if the detected amount of pepsinogen II is equal to or greater than 20 μg/L and the Helicobacter pylori status is positive;
b) an intermediate-risk group for early-stage diffuse-type gastric cancer if the detected amount of pepsinogen II is equal to or greater than 20 μg/L or the Helicobacter pylori status is positive; and
c) a low-risk group for early-stage diffuse-type gastric cancer if the detected amount of pepsinogen II is less than 20 μg/L and the Helicobacter pylori status is negative.

15. (canceled)

16. (canceled)

17. (canceled)

18. The method according to claim 1, wherein
wherein method comprises predicting or diagnosing a subject to have early-stage diffuse-type gastric cancer if the level of pepsinogen II is 20 μg/L.

19. The method according to claim 1,
wherein the method further comprises measuring whether the subject is infected with Helicobacter pylori (HP).

20. The method according to claim 19,
wherein the measurement of whether the subject is infected with Helicobacter pylori comprises one or more selected from the group consisting of a histological test, a culture test, a rapid urease test, and a serological test,
the histological test is a staining test on a biopsy specimen isolated from stomach of the subject,
the culture test is a culture test of Helicobacter pylori on a biopsy specimen isolated from stomach of the subject, and
the serological test is to measure immunoglobulin G (IgG), an antibody against Helicobacter pylori, in serum of the subject.

21. The method according to claim 20,
wherein the method comprises classifying a Helicobacter pylori (HP) status of a subject as a positive or negative status according to the following criteria:
i) the Helicobacter pylori status is positive if one or more selected from the group consisting of the histological test, culture test, rapid urease test, and serological test are positive;
ii) the Helicobacter pylori status is positive if the histological test, culture test, and rapid urease test are all negative and the serological test is positive;
iii) the Helicobacter pylori status is positive if the histological test, culture test, rapid urease test, and serological test are negative and the subject has a history of eradication of Helicobacter pylori; and
iv) the Helicobacter pylori status is negative if the histological test, culture test, rapid urease test, and serological test are negative and the subject does not have a history of eradication of Helicobacter pylori, and
the subject is predicted or diagnosed to be any one of a high-risk group, an intermediate-risk group, or a low-risk group for early-stage diffuse-type gastric cancer depending on a level of Pepsinogen II and a Helicobacter pylori status of the subject:
a) a high-risk group for early-stage diffuse-type gastric cancer if the detected amount of pepsinogen II is equal to or greater than 20 μg/L and the Helicobacter pylori status is positive;
b) an intermediate-risk group for early-stage diffuse-type gastric cancer if the detected amount of pepsinogen II is equal to or greater than 20 μg/L or the Helicobacter pylori status is positive; and
c) a low-risk group for early-stage diffuse-type gastric cancer if the detected amount of pepsinogen II is less than 20 μg/L and the Helicobacter pylori status is negative.
